(19) United States
(12) Patent Application Publication
(10) Pub. No.: US 2021/0088536 A1
(43) Pub. Date: Mar. 25, 2021

(54) Blood Clot Indicator Test and Device

(71) Applicant: Nathan D. Wetherell, Leadville, CO (US)

(72) Inventor: Nathan D. Wetherell, Leadville, CO (US)

(21) Appl. No.: 16/948,535

(22) Filed: Sep. 23, 2020

Related U.S. Application Data
(60) Provisional application No. 62/904,930, filed on Sep. 24, 2019.

(51) Int. Cl. G01N 33/86; G01N 33/543

(52) U.S. Cl. CPC G01N 33/86; G01N 33/54306

(57) ABSTRACT

A blood clot indicator test and device enables rapid detection of clots. An example device for a blood clot indicator test includes a sample pad configured to receive a blood serum. One or more test lines configured with an analyte. Different analytes may be provided, each corresponding to separate bodily sites. The test lines can be read to indicate whether the blood serum is affected by a clot at one or more separate bodily sites.

### PRIORITY CLAIM

This application claims the priority filing benefit of U.S. Provisional Patent Application No. 62/904,930 filed Sep. 24, 2019 titled “Blood Clot Indicator Test and Device” of Nathan D. Wetherell, hereby incorporated herein by reference in its entirety as though fully set forth herein.

### BACKGROUND

The human body has an endogenous system intertwined with the central nervous system, lymphatic system, and cardiovascular/circulatory system that regulates these crucial bodily systems and functions and plays a key role in neuro and cardiac homeostatic physiology. This system is known as the Endogenous Cannabinoid System.

It has been shown that the Endogenous Cannabinoid System, and the Endo-cannabinoids this system produces, impact the functionality and survival of the heart, lungs and brain following a blockage resulting in ischemia. It has also been shown that cannabinoids play a key role in heart health and that the heart and lungs have a direct and immediate reaction to such cannabinoids whether they are plant (phyto) or bodily (endo) in origin.

### BRIEF DESCRIPTION OF THE DRAWINGS

FIG. 1 shows an example device for a blood clot indicator test.

FIG. 2 shows another example device for a blood clot indicator test.

FIGS. 3A-3C illustrate example operation of the blood clot indicator test and device.

### DETAILED DESCRIPTION

An example blood clot indicator test and device are disclosed which enable faster detection methods of cerebral and cardiac infarcts. This is important as a source for a point of contact (POC), real-time detection method for identifying, diagnosing, and treating blood clots and related conditions.

The human body (and perhaps animals) has an Endogenous Cannabinoid System that regulates homeostasis in the human body. When a vascular or cerebral infarct or event occurs resulting in ischemia or related condition, the body releases endogenous cannabinoids into the bloodstream. These cannabinoids and their plant based (phyto) counterparts have been proven to have homeostatic effects on the heart, and protective properties that prevent oxidation and cellular death in the brain. These cannabinoids, and their plant-based (phyto) and synthetic counterparts are referred to as neuro-protectants.

It is believed that the body produces endocannabinoids en masse, as an attempt to keep the body in homeostatic range as a response to a blockage in the circulatory system, e.g., from a blood infarct or a buildup of plaque. When a vascular event occurs, the body releases endogenous cannabinoids to protect vital organs from ischemic damage.

These endogenous cannabinoids can be detected in the blood, saliva, sweat, urine and breath analysis. Rapid detection as disclosed herein, utilizes xeno biological material as detection analytes. These endogenous cannabinoids, when detected at certain thresholds, can be implemented by a treating physician and/or other involved in treatment to rapidly predict the likelihood that a patient is suffering a heart attack or stroke or other health issue associated with the blockage. In an example, the test may implement precise detection methods to help a treating physician and/or others involved in treatment to pinpoint the location(s) of the infarct.

In an example, the test disclosed herein may be implemented as a Lateral Flow Assay (LFA) test or an ELISA test. A patient's blood serum (e.g., following a finger prick or venous puncture) is collected directly onto an analyte pad having one or more test lines. Test lines include activating particles that indicate whether the patient is experiencing a blockage.

In an example, the test can be used by physicians or others involved in treatment, such as but not limited to, paramedics and other first responders. The test may also be provided as a clot indicator for use by patients themselves (e.g., cardiac or circulatory patients prescribed anticoagulants such as Heparin™ or Coumadin™).

Before continuing, it should be noted that the examples described above are provided for purposes of illustration, and are not intended to be limiting. Other devices and/or device configurations may be utilized to carry out the operations described herein.

It is also noted that as used herein, the terms “includes” and “including” mean, but is not limited to, “includes” or “including” and “includes at least” or “including at least.” The term “based on” means “based on” and “based at least in part on.”

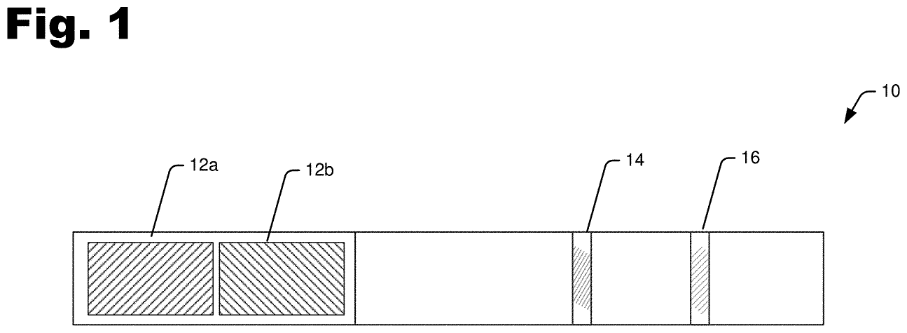

FIG. 1 shows an example device 10 fora blood clot indicator test. In an example, the test device 10 includes a Lateral Flow Assay. Blood is applied to the sample pad 12a and/or 12b. Pad 12b may include bodily specific references, as explained in more detail below. The blood serum flows to the test line 14 and the control line 16. The test line 14 and control 16 indicate a positive or a negative as the blood flows and interacts with the analyte.

The test device 10 is not limited to any particular type of assay. Nor is the device 10 limited to any number of test and/or control lines. For example, the test may include multiple test lines.

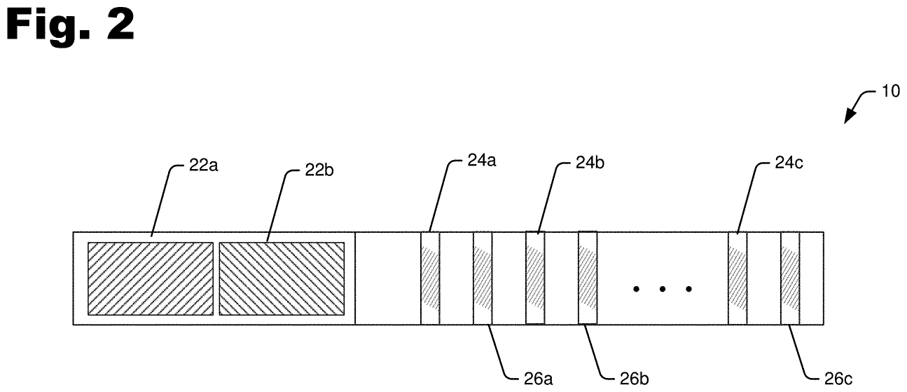

FIG. 2 shows another example device 20 for a blood clot indicator test. The test device 20 includes a sample pad or other collection area 22, a plurality of test lines 24a-c, and corresponding control lines 26a-c.

In an example, each test line 24a-c may be specific to a designated bodily site (e.g., Heart, Lungs, Brain and Peripheral). These test lines 24a-c indicate which major area of the body is being affected by the clot, as determined by the analyte and/or amount of analyte provided at each test line 24a-c. For example, the test lines 24a-c may each include a separate endocannabinoid, agonist, and/or antagonist from a specific tissue or chemical agent sourced material. As such, testing by separate types of analytes (e.g., based on originating tissue or chemical agent) indicates more specifically where in the body the blockage is occurring. For example, if test lines 24a (e.g., Heart), and 24b (Lungs) are negative, but test line 24c (e.g., Brain) is positive, then the test indicates that the clot may be occurring in the brain.

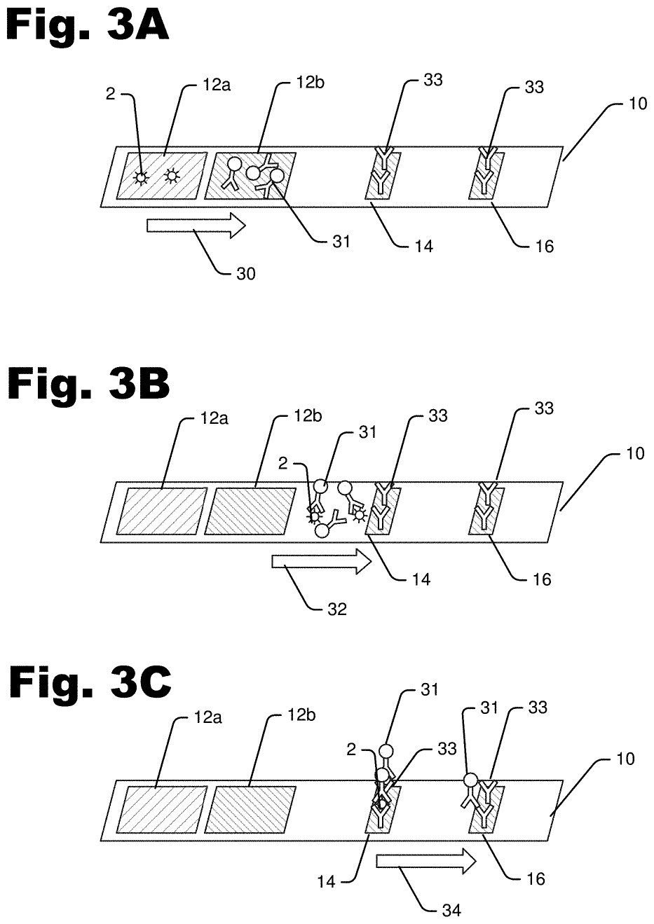

FIGS. 3A-3C illustrate example operation of the blood clot indicator test and device. In an example, the blood clot indicator test includes receiving a blood serum 2 on the test device. A test device similar to the test device 10 is shown in FIGS. 3A-3C. However, it is noted that similar operations would also apply for test device 20, and for other types of test devices such as electronic devices.

The sample (e.g., blood) may be applied to the sample pad 12a in the operation referred to by reference 30 in FIG. 3A. It is noted that blood is used by way of illustration herein, however, other samples (e.g., saliva) may also be utilized.

In an example, the blood serum moves across pad 12b in an operation referred to by reference 32 in FIG. 3B. For example, the pad 12b may include an endocannabinoid 31 from a specific tissue or chemical agent sourced material, which will aid in determining where in the body the blockage is occurring.

In an example, a plurality of test lines each include an agonist and/or antagonist from a specific tissue or chemical agent sourced material, thereby indicating where in the body the blockage is occurring (e.g., Heart, Lung, Brain, Peripheral). In an example, the test device 10 implements endogenous cannabinoids (e.g., animals), phyto-cannabinoids (e.g., from plant material), and synthetic cannabinoids (e.g., man-made materials) and/or endogenous, phyto, or synthetic cannabinoid receptors as an agonist/antagonist analyte for testing.

In FIG. 3C, the blood serum in combination with the originating tissue or chemical agent moves to one or more test lines in an operation referred to by reference 34 in FIG. 3C. A single test line 14 is shown for purposes of illustration, but the test device may include multiple test lines.

The test line(s) 14 are configured with a cannabinoid and/or cannabinoid receptor analyte 33. The control line(s) 16 may also include the same cannabinoid and/or cannabinoid receptor analyte 33. Interaction with the analyte on the test line 14 and control line 16 results in a positive or negative (when the test line 14 is compared to the control line 16) to indicate whether the blood serum is affected by a clot.

In an example, a positive result appearing at the test line 14 and corresponding with the control line 16 indicates a clot. If a specific test line 14 (or test lines) are provided, the test lines which are positive indicate where in the body the blockage is occurring (e.g., Heart, Lung, Brain Peripheral). Based on the test results indicated by the test device, the correct medications can be administered in a timely manner.

The operations shown and described herein are provided to illustrate example implementations. It is noted that the operations are not limited to the ordering shown. Still other operations may also be implemented.

In an example, the test device (e.g., device 10 or 20) includes cannabinoid and/or cannabinoid receptor as the analyte. The test device is unlike any other device for detecting infarcts at such a rapid response (minutes versus hours or days). The test device 10 may function to test multiple bodily organs and systems from one blood test and indicate a clot or blockage at designated sites, e.g., for heart, lungs, brain, kidney, liver and peripheral tissues.

In recent years the investigation into the Endogenous Cannabinoid System has grown exponentially. In publications it has become clear that the body produces its own cannabinoids in an effort to maintain homeostasis. The production of endogenous cannabinoids by the human body was discovered and presented to the scientific community by Raphael Melchoulam of Hebrew University. In the following years, Robert Devane of Washington University discovered the abundant amount of cannabinoid 1 and 2 receptors present in rat brain models. Devane's discovery along with Dr. Melchoulam's indicates that the body has a Cannabinoid System and is called the Endogenous Cannabinoid System or ECS. This system not only has receptors, but also produces endocannabinoids that directly affect the CB1/2 receptors. These endogenous cannabinoids mimic \hat{ }8,9THC, CBD, CBN, THCV, CBDA, THCA, CBC and many other cannabinoids commonly found in the Narrow Leaf Drug Cannabis Sativa Chemotype of the Hemp plant.

The body produces endocannabinoids. The heart and brain have an abundant amount of cannabinoid receptors identified as receptors 1 and 2. Receptors 1 and 2 are activated by the presentation of either THC, THC/CBD or CBN/CBD and many other combinations of cannabinoids and endogenous cannabinoids. It is noted that certain cannabinoids have a therapeutic, protective, and regenerative effect on the Circulatory system and the central nervous system.

These E-cannabinoids are often produced when there are physiological changes to the human body, such as dehydration, loss of oxygen to the muscles, or a loss of oxygen to the brain. These studies have also proven that the heart creates endogenous cannabinoids in the walls of the cardiac muscle, helping with circulation, proper vascular electrical conduction through the AV, SV nodes, and helps support vascular contractility, functionality and protects and preserves the heart, lungs and brain from damage due to ischemia.

In the lungs, cannabinoids play a key role in pulmonary function, this includes bronchial dilation, and protection of the alveoli. It is of note that there is currently a patent on \hat{ }9-THC as a potent neuro protectant against ischemia or loss of oxygen to the brain. This enables the test and test device disclosed herein to utilize the E-cannabinoid system as an indicator for cardiac, pulmonary or cerebral infarcts and or blockages.

More specifically, if a patient is experiencing a cardiac/pulmonic or neurological event caused by the production of an infarct, that by using CB1 and CB2 receptor rich tissue in a Lateral Flow Assay or other to be determined assay techniques, these certain E-cannabinoids can be detected, and rapidly indicate via blood serum draw, whether a patient is experiencing a cardiac, pulmonary or cerebral infarct.

In other words, medical personnel can detect a heart attack or stroke in minutes without the need for a CT, MRI, or a heart cath. This test is non-invasive, non-surgical and may not even require a doctor's order to perform.

In an example, the test device may include electronic detection and be similar in size to a glucose monitor. As such, an EMT and other first responders can accurately detect a stroke, heart attack or any other blockage before arriving at the Emergency Department. This test may also improve patient survivability and reduce damage to the brain, heart and lungs.

In multiple studies performed on mouse hearts, CB1/2 agonists JWH-133, HU-210 were injected into the heart before being re-perfused. The synthetic cannabinoid JWH-133 showed a protective effect on the mouse heart and also reduced infarct size as well.

There may also be many more cannabinoid receptors present in the heart, yet to be discovered. These are referred to herein as Intra-Cardia-Cannabinoid receptors or ICCBr. With the activation of these ICCBr's, protection against ischemia from a stroke is activated.

Thus, by utilizing the this information, medical care providers can use the tests and test devices disclosed herein to pinpoint the endogenous cannabinoids and the corresponding agonistic/antagonistic peripheral tissue which then act as an indicator for an ELISA or a Lateral Flow Assay (LFA). By using mouse, canine, porcine, rat, heart lung and brain material, an abundance of anandamide and 2-arachidonolyl glycerol along with other E-cannabinoids can be detected and indicate a possible stroke, pulmonary emboli or heart attack.

THC and CBD have also shown promise in the reduction of plaque from atherosclerosis. This is also another indicator that the ECS responds to infarcts by releasing endocannabinoids into the circulatory system as a means of protection and lubrication of the blood. This lubrication could also be a clot busting action.

Endogenous cannabinoids play a role in cardiac and cerebral function in ischemic dysfunctions. This in turn plays a key role in detecting such infarcts.

The test device disclosed herein may also result in stroke and heart attack diagnosis turnaround time in terms of minutes (rather than hours, according to current laboratory techniques). In the past, heart attacks and strokes were diagnosed using a myriad of tests with no conclusive answer, just a collection of symptoms. The test device disclosed herein can detect the potential for an infarct much faster (e.g., within minutes). Accordingly, EMS and other first responders may employ these in their emergency kits.

The test device has utility in every part of the world, for example in Emergency Departments, Urgent Care, health clinics and emergency response vehicles. The test device utilizes animal, plant, human tissues and synthetic materials in cannabinoids and/or cannabinoid receptors to detect human circulatory system blockages.

It is noted that the examples shown and described are provided for purposes of illustration and are not intended to be limiting. Still other examples are also contemplated.

1. A device for a blood clot indicator test, comprising:
a sample pad configured to receive a blood serum;
at least one test line for receiving the blood serum from the sample pad, the at least one test line configured with an analyte; and
wherein the blood serum flows to the at least one test line, the at least one test line resulting in a positive or a negative to indicate whether the blood serum is affected by a clot.

2. The device of claim 1, further comprising at least one control line for receiving the blood serum from the sample pad and comparing to the result at the at least one test line, wherein a positive on the at least one control line indicates a clotting issue.

3. The device of claim 1, wherein the at least one test line includes at least one cannabinoid receptor as the analyte.

4. The device of claim 1, wherein the at least one test line includes an endocannabinoid from a specific tissue or chemical agent sourced material, thereby indicating where in the body the blockage is occurring.

5. The device of claim 1, wherein the at least one test line includes an agonist from a specific tissue or chemical agent sourced material, thereby indicating where in the body the blockage is occurring.

6. The device of claim 1, wherein the at least one test line includes an antagonist from a specific tissue or chemical agent sourced material, thereby indicating where in the body the blockage is occurring.

7. The device of claim 1, wherein the positive on the at least one control line enables detecting infarcts for rapid response.

8. The device of claim 1, wherein the analyte includes endogenous cannabinoids and/or cannabinoid receptors from animals.

9. The device of claim 1, wherein the analyte includes phyto-cannabinoids and/or phyto-cannabinoid receptors from plants.

10. The device of claim 1, wherein the analyte includes synthetic cannabinoids and/or cannabinoid receptors.

11. The device of claim 1, wherein the analyte is specific to at least one bodily organ.

12. The device of claim 1, wherein the analyte is specific to multiple different bodily sites, and wherein a single blood test indicates a clot at one or more of the multiple different bodily sites.

13. The device of claim 12, wherein the multiple different bodily sites include at least heart, lungs, brain, kidney, liver and peripheral tissues.

14. A device for a blood clot indicator test, comprising:
a sample pad configured to receive a blood serum;
a plurality of test lines for receiving the blood serum from the sample pad, the plurality of test lines each configured with a different analyte, each of the different analytes corresponding to separate bodily sites; and
wherein the blood serum flows to the plurality of test lines to indicate whether the blood serum is affected by a clot at one or more separate bodily sites.

15. The device of claim 14, further comprising a control line for each of the plurality of test lines.

16. The device of claim 14, wherein the plurality of test lines each include at least one cannabinoid and/or cannabinoid receptor as the analyte.

17. The device of claim 14, wherein the plurality of test lines each includes an endocannabinoid, agonist, and/or antagonist from a specific tissue or chemical agent sourced material, thereby indicating where in the body the blockage is occurring.

18. A blood clot indicator test, comprising:
receiving a sample to be tested;
providing the sample onto a plurality of different cannabinoid and/or cannabinoid receptor analytes each corresponding to separate bodily sites; and
wherein the sample interacts with the plurality of different cannabinoid and/or cannabinoid receptor analytes to indicate whether the blood serum is affected by a clot at one or more separate bodily sites.

19. The blood clot indicator test of claim 18, wherein the plurality of different cannabinoid and/or cannabinoid receptor analytes include an endocannabinoid, agonist, and/or antagonist from a specific tissue or chemical agent sourced material, thereby indicating where in the body the blockage is occurring.

20. The blood clot indicator test of claim 18, wherein the plurality of different cannabinoid and cannabinoid and/or cannabinoid receptor analytes include cannabinoids based on plants, animals, and synthetic cannabinoid materials.
